(19) United States
(12) Patent Application Publication
(10) Pub. No.: US 2007/0232565 A1
(43) Pub. Date: Oct. 4, 2007

(54) NOVEL POLYSACCHARIDES AND OLIGOSACCHARIDES

(75) Inventor: Kazuyuki SUGAHARA, Kyoto (JP)

Correspondence Address: OBLON, SPIVAK, MCCLELLAND, MAIER & NEUSTADT, P.C., 1940 DUKE STREET, ALEXANDRIA, VA, 22314, US

(73) Assignees: Kazuyuki SUGAHARA, Kyoto-shi (JP); MARUHA CORPORATION, Chiyoda-ku (JP)

(21) Appl. No.: 11/694,075

(22) Filed: Mar. 30, 2007

(30) Foreign Application Priority Data
Mar. 31, 2006 (JP) 2006-098722
Oct. 2, 2006 (JP) 2006-270911

(51) Int. Cl. A61K 31/737; C08B 37/00

(52) U.S. Cl. 514/54; 536/54

(57) ABSTRACT

Polysaccharides and oligosaccharides having activity of binding to different proliferation factors and neurite outgrowth-promoting activity and compositions for neurological disease treatment, compositions for inflammatory disease treatment, or compositions for wound treatment, which comprise such polysaccharides and oligosaccharides, are provided. Chondroitin sulfate/dermatan sulfate hybrid chains, chondroitin sulfate C, and oligosaccharides derived from either thereof are provided.

### BACKGROUND OF THE INVENTION

1. Field of the Invention

The present invention relates to novel polysaccharides and oligosaccharides. In particular, the present invention relates to shark-derived polysaccharides and oligosaccharides.

2. Background Art

Animal tissue contains a group of sulfated sugar chains, which are referred to as glycosaminoglycans. Such sulfated sugar chains exist as proteoglycan molecules covalently linked to proteins and play essential roles in cell growth and differentiation, tissue morphogenesis, and the like (see Non-Patent Documents 1 to 4).

Among glycosaminoglycans, chondroitin sulfate is composed of a disaccharide (D-glucuronic acid and N-acetyl-D-galactosamine) and a sulfate residue. In addition, chondroitin sulfate is classified into types such as A, C, D, E, and K according to the binding position of the sulfate groups. Chondroitin sulfate B contains L-iduronic acid instead of D-glucuronic acid as a constituent sugar and is referred to as dermatan sulfate.

Chondroitin sulfate and dermatan sulfate contain many overlapping sequences composed of disaccharide units modified by different sulfation patterns, exhibiting enormous structural diversity comparable to heparan sulfate.

The present inventors have tried to elucidate the involvement of a variety of oversulfated chondroitin sulfate and dermatan sulfate chains in neural development. As a result, the present inventors have taken notice of the importance of oversulfated disaccharide units such as the “D” unit [GlcUA(2S)-GalNAc(6S)], the “iD” unit [IdoUA(2S)-GalNAc(6S)], the “E” unit [GlcUA-GaINAc(4S,6S)], and the “iE” unit [IdoUA-GalNAc(4S,6S)] in chondroitin sulfate and dermatan sulfate having various biological functions (wherein IdoUA represents L-iduronic acid, GlcUA represents D-glucuronic acid, GalNAc represents N-acetyl-D-galactosamine, and 2S, 4S, and 6S represent the binding sites of the sulfate group at the C-2 position, the C-4 position, and the C-6 position, respectively).

Each neuron develops a characteristic morphology during the formation of a neural circuit while extending its axon along a specific pathway. Accordingly, each neuron finds a correct target cell so as to form a synapse therewith. Many researchers have previously found that the chondroitinase ABC treatment of brain tissue in the developmental stage to decompose and remove chondroitin sulfate contained in the tissue causes severe abnormalities in neuronal morphogenesis and axonal course. Based on such observation, chondroitin sulfate has been thought to be involved in the induction of axons of neurites or in neuronal morphogenesis.

The inventors of the present invention cultured hippocampal neurons prepared from embryonic day 16 mice on substrates coated with a variety of chondroitin sulfate (dermatan sulfate) preparations. Thus, they have found that sugar chains having particular structures exhibit neurite outgrowth-promoting activity. In particular, the present inventors have found that such sugar chains containing a D or iD unit in a large amount enable extension of dendrite-like projections while those rich in an E or iE unit enable extension of long axon-like projections (see Patent Document 1 and Non-Patent Documents 5 and 6).

Such oversulfated chondroitin sulfate and dermatan sulfate are known to bind to many heparin-binding growth factors, suggesting that signal transduction relating to such growth factors is involved in the morphogenesis of hippocampal neurons.

Based on these facts, it has been expected to search a supply source of glycosaminoglycan having a unique structure and remarkable activity, which is useful as a therapeutic agent for neurological diseases. As already reported, mucopolysaccharide has been purified from the skins of marine organisms such as sharks (see Non-Patent Document 7).

[Patent Document 1] WO2005/103089

[Non-Patent Document 1] Lewandowska, K., Choi, H. U., Rosenberg, L. C., Zardi, L., and Culp, L. A. (1987) J. Cell Biol. 105, 1443-1454

[Non-Patent Document 2] Yamaguchi, Y., Mann, D. M., and Ruoslahti, E. (1990) Nature, 346, 281-284

[Non-Patent Document 3] Lyon, M., Deakin, J. A., Rahmoune, H., Fernig, D. G., Nakamura, T., and Gallagher, J. T. (1998) J. Biol. Chem. 273, 271-278

[Non-Patent Document 4] Trowbridge, J. M. and Gallo, R. L. (2002) Glycobiology 12, 117R-125R

[Non-Patent Document 5] Hikino, M., Mikami, T., Faissner, A., Vilela-Silva, A. C., Pavao, M. S., and Sugahara, K. (2003) J. Biol. Chem. 278, 43744-43754

[Non-Patent Document 6] Sugahara, K. and Yamada, S., Trends in Glycoscinence and Glycotechnology vol. 12 No. 67 pp.321-349

[Non-Patent Document 7] Seno, N. and Meyer, K., Biochim. Biophys. Acta. 78 (1963) 258-264

### SUMMARY OF THE INVENTION

It is an objective of the present invention to provide polysaccharides and oligosaccharides having activity of binding to a variety of proliferation factors and neurite outgrowth-promoting activity. It is another objective of the present invention to provide a composition for neurological disease treatment, a composition for inflammatory disease treatment, or a composition for wound treatment.

The inventors of the present invention have found that polysaccharide and oligosaccharide fractions that have been separated and purified from tissue of a part of a shark bind to a variety of proliferation factors. Further, they have found that such fractions have neurite outgrowth-promoting activity and anticoagulant activity, and that such fractions have effects as compositions for neurological disease treatment, compositions for inflammatory disease treatment, and compositions for wound treatment. This has led to the completion of the present invention. Thus, the present invention relates to the following embodiments:
1. oligosaccharides derived from chondroitin sulfate/dermatan sulfate hybrid chains, comprising sulfated hexasaccharide structures and being resistant to chondroitinase AC-I, wherein the oligosaccharides are derived from a chondroitin sulfate/dermatan sulfate hybrid;
2. the oligosaccharides according to 1 above, wherein the oligosaccharides are derived from chondroitin sulfate/dermatan sulfate hybrid chains extracted from shark skin;
3. the oligosaccharides derived from chondroitin sulfate/dermatan sulfate hybrid chains according to 1 or 2 above, wherein the oligosaccharides have neurite outgrowth-promoting activity and bind to pleiotrophin;
4. oligosaccharides derived from chondroitin sulfate/dermatan sulfate hybrid chains, wherein the oligosaccharides are represented as ΔHexUAα1-3GalNAc(4S)β1-4IdoUA(2S)α1-3GalNAc(4S)β1-4IdoUA(2S)α1-3GaINAc(4S);
5. a chondroitin sulfate/dermatan sulfate hybrid chain comprising a structure represented as ΔHexUAα1-3GalNAc(4S)β1-4IdoUA(2S)α1-3GalNAc(4S)β1-4IdoUA(2S)α1-3GalNAc(4S);
6. oligosaccharides derived from chondroitin sulfate C, comprising a sulfated hexasaccharide structure and being resistant to chondroitinase AC-I;
7. the oligosaccharides according to 6 above, wherein the oligosaccharides areiderived from chondroitin sulfate C extracted from shark cartilage;
8. the oligosaccharides derived from chondroitin sulfate C according to 6 or 7 above, wherein the oligosaccharides bind to hepatocellular growth factors, midkine, pleiotrophin, and RANTES;
9. oligosaccharides derived from chondroitin sulfate C, comprising a structure selected from the group consisting of ΔHexUAα1-3GalNAc(4S)β1-4GlcUAβ1-3GalNAc(4S)β1-4GlcUA(2S)β1-3GalNAc(6S)(ΔA-A-D), ΔHexUAα1-3GalNAc(6S)β1-4GlcUA(2S)β1-3GalNAc(6S)β1-4GlcUAβ1-3GalNAc(6S) (ΔC-D-C), ΔHexUAα1-3GalNAc(4S)β1-4GlcUA(2S)β1-3GalNAc(6S)β1-4GlcUAβ1-3 GalNAc(4S) (ΔA-D-A), ΔHexUAα1-3 GalNAc(4S)β1-4GlcUA(2S)β1-3GalNAc(6S)β1-4GlcUA(2S)β1-3GalNAc(6S) (ΔA-D-D), ΔHexUAα1-3GalNAc(6S)β1-4GlcUA(2S)β1-3GalNAc(6S)β1-4GlcUA(2S)β1-3GalNAc(6S) (ΔC-D-D), ΔHexUAα1-3GalNAc(4S) β1-4GlcUA(2S)β1-3GalNAc(6S)β1-4GlcUAβ1-3GalNAc(6S) (ΔA-D-C), ΔHexUAα1-3GalNAc(4S 6S)β1-4GlcUA β1-3 GalNAc(4S)β1-4GlcUA(2S)β1-3GalNAc(6S) (ΔE-A-D), ΔHexUA(2S)α1-3GalNAc(6S)β1-4GlcUA(2S)β1-3GalNAc(6S)βB1-4GlcUAβ1-3GalNAc(6S) (ΔD-D-C), ΔHexUAα1-3GalNAc(4S,6S)β1-4GlcUA(2S)β1-3GalNAc(6S)β1-4GlcUA β1-3GalNAc(6S) (ΔE-D-C), ΔHexUAα1-3GalNAc(6S)β1-4GlcUA1-3GalNAc(4S)β1-4GlcUA(2S)β1-3GalNAc(6S) (ΔC-A-D), ΔHexUAα1-3GalNAc(4S)β1-4GlcUAβ1-3GalNAc(4S,6S)β1-4GlcUA(2S)β1-3GalNAc(6S) (ΔA-E-D), ΔHexUAα1-3GalNAc(4S)β1-4GlcUA(2S)β1-3GalNAc(4S)β1-4GlcUA(2S)β1-3GalNAc(6S) (ΔA-B-D), ΔHexUA(2S)α1-3GalNAc(6S)β1-4GlcUA(2S)β1-3 GalNAc(6 S)β1-4GlcUA(2S)β1-3 GalNAc(6S) (ΔD-D-D), ΔHexUAα1-3GalNAc(4S,6S)β1-4GlcUA(2S)β1-3GalNAc(6S)β1-4GlcUA(2S)β1-3GalNAc(6S) (ΔE-D-D), and ΔHexUAα1-3GalNAc(4S)β1-4GlcUA(2S)β1-3GalNAc(6S)β1-4GlcUA(2S)β1-3GalNAc(4S,6S) (ΔA-D-T) or ΔHexUA(2S)α1-3GalNAc(6S)β1-4GlcUAβ1-3GalNAc(4S)β1-4GlcUA(2S)β1-3GalNAc(4S,6S) (ΔD-A-T);
10. chondroitin sulfate C comprising a structure of the oligosaccharide derived from chondroitin sulfate C according to 9 above;
11. chondroitin sulfate/dermatan sulfate hybrid chains comprising at least one unit selected from the group consisting of Δ^{4,5}HexUAα1-3GalNAc(ΔO), GlcUAβ1-3GalNAc(O), Δ^{4,5}HexUAα1-3GalNAc(4S)(ΔA), GlcUAβ1-3GalNAc(4S)(A), Δ^{4,5}HexUAα1-3GalNAc(6S)(ΔC), GlcUAβ1-3GalNAc(6S)(C), IdoUAα1-3GalNAc(6S)(iC), Δ^{4,5} HexUA(2S)α1-3GalNAc(4S)(ΔB), GlcUA(2S)β1-3GalNAc(4S)(B), IdoUA(2S)α1-3GalNAc(4S)(iB), Δ^{4,5}HexUA(2S)α1-3GalNAc(6S)(ΔD), GlcUA(2S)β1-3GalNAc(6S)(D), IdoUA(2S)α1-3GalNAc(6S)(iD), Δ^{4,5}HexUAα1-3GalNAc(4S,6S)(ΔE), GlcUAβ1-3GalNAc(4S,6S)(E), IdoUAα1-3GalNAc(4S,6S)(iE), Δ^{4,5}HexUA(2S)α1-3GalNAc(4S,6S)(ΔT), and GlcUA(2S)β1-3GalNAc(4S,6S)(T);
12. the chondroitin sulfate/dermatan sulfate hybrid chains according to 11 above, wherein the chains are derived from shark liver;
13. the chondroitin sulfate/dermatan sulfate hybrid chains according to 11 or 12 above, wherein the chains have neurite outgrowth-promoting activity and bind to fibroblast growth factor-2, fibroblast growth factor-7, heparin-binding epidermal cell growth factor, vascular endothelial growth factor, hepatocellular proliferation factors, midkine, and pleiotrophin;
14. An agent for binding a growth factor or a cytokine comprising, as an active ingredient, at least one oligosaccharide, chondroitin sulfate/dermatan sulfate hybrid chain, or chondroitin sulfate C selected from the group consisting of the oligosaccharide according to any one of 1 to 4 above, the chondroitin sulfate/dermatan sulfate hybrid chain according to 5 above, the oligosaccharide according to any one of 6 to 9 above, the chondroitin sulfate C according to 10 above, and the chondroitin sulfate/dermatan sulfate hybrid chain according to any one of 11 to 13 above;
15. An agent for promoting a growth factor or cytokine activity comprising, as an active ingredient, at least one oligosaccharide, chondroitin sulfate/dermatan sulfate hybrid chain, or chondroitin sulfate C selected from the group consisting of the oligosaccharide according to any one of 1 to 4 above, the chondroitin sulfate/dermatan sulfate hybrid chain according to 5 above, the oligosaccharide according to any one of 6 to 9 above, the chondroitin sulfate C according to 10 above, and the chondroitin sulfate/dermatan sulfate hybrid chain according to any one of 11 to 13 above;
16. An agent for promoting neurite outgrowth comprising, as an active ingredient, at least one oligosaccharide, chondroitin sulfate/dermatan sulfate hybrid chain, or chondroitin sulfate C selected from the group consisting of the oligosaccharide according to any one of 1 to 4 above, the chondroitin sulfate/dermatan sulfate hybrid chain according to 5 above, the oligosaccharide according to any one of 6 to 9 above, the chondroitin sulfate C according to 10 above, and the chondroitin sulfate/dermatan sulfate hybrid chain according to any one of 11 to 13 above;
17. a pharmaceutical composition comprising, as an active ingredient, at least one oligosaccharide, chondroitin sulfate/dermatan sulfate hybrid chain, or chondroitin sulfate C selected from the group consisting of oligosaccharide according to any one of 1 to 4 above, chondroitin sulfate/dermatan sulfate hybrid chain according to 5 above, the oligosaccharide according to any one of 6 to 9 above, the chondroitin sulfate C according to 10 above, and the chondroitin sulfate/dermatan sulfate hybrid chain according to any one of 11 to 13 above;
18. a pharmaceutical composition for prevention or treatment of neurological diseases, inflammatory diseases, or wounds comprising, as an active ingredient, at least one oligosaccharide, chondroitin sulfate/dermatan sulfate hybrid chain, or chondroitin sulfate C selected from the group consisting of the oligosaccharide according to any one of 1 to 4 above, the chondroitin sulfate/dermatan sulfate hybrid chain according to 5 above, the oligosaccharides according to any one of 6 to 9 above, the chondroitin sulfate C according to 10 above, and the chondroitin sulfate/dermatan sulfate hybrid chain according to any one of 11 to 13 above;
19. use of at least one oligosaccharide, chondroitin sulfate/dermatan sulfate hybrid chain, or chondroitin sulfate C selected from the group consisting of the oligosaccharide according to any one of 1 to 4 above, the chondroitin sulfate/dermatan sulfate hybrid chain according to 5 above, the oligosaccharide according to any one of 6 to 9 above, the chondroitin sulfate C according to 10 above, and the chondroitin sulfate/dermatan sulfate hybrid chain according to any one of 11 to 13 above for production of a growth factor-binding agent, a neurite outgrowth-promoting agent, a composition for neurological disease treatment, a composition for inflammatory disease treatment, or a composition for wound treatment; and
20. use of at least one oligosaccharide, chondroitin sulfate/dermatan sulfate hybrid chain, or chondroitin sulfate C selected from the group consisting of the oligosaccharide according to any one of 1 to 4 above, the chondroitin sulfate/dermatan sulfate hybrid chain according to 5 above, the oligosaccharide according to any one of 6 to 9 above, the chondroitin sulfate C according to 10 above, and the chondroitin sulfate/dermatan sulfate hybrid chain according to any one of 11 to 13 above for production of a growth factor activity-promoting agent.

According to the present invention, an oligosaccharide, chondroitin sulfate/dermatan sulfate hybrid chain, or chondroitin sulfate C, which binds to a variety of proliferation factors and exhibits neurite outgrowth-promoting activity, is provided. Further, a composition for neurological disease treatment, a composition for inflammatory disease treatment, or a composition for wound treatment, comprising any of the above, is provided. In addition, the oligosaccharide, chondroitin sulfate/dermatan sulfate hybrid chain, or chondroitin sulfate C of the present invention is derived from sharks. Thus, based on the present invention, it has been found that fish can be used for useful applications.

### BRIEF DESCRIPTION OF THE DRAWINGS

FIG. 1 shows activity of the oligosaccharide of the present invention to inhibit binding of a hepatocyte growthfactor (HGF) to embryonic pig brain-derived CS/DS (E-CS/DS).

FIG. 2 shows activity of the oligosaccharide of the present invention to inhibit binding of midkine (MK) to E-CS/DS.

FIG. 3 shows activity of the oligosaccharide of the present invention to inhibit binding of RNATES to E-CS/DS.

FIG. 4 shows activity of the oligosaccharide of the present invention to inhibit binding of RNATES to shark cartilage-derived CS-D.

FIG. 5 shows activity of the oligosaccharide of the present invention to inhibit binding of pleiotrophin (PTN) to shark cartilage-derived CS-D.

FIG. 6 shows interaction between a variety of growth factors and the polysaccharides of present invention and the enzyme digests thereof.

FIG. 7 shows interaction between pleiotrophin (PTN) and polysaccharides of the present invention and the enzyme digests thereof.

FIG. 8 shows neurite outgrowth promoting activities of liver-derived chondroitin sulfate/dermatan sulfate hybrid chains of the present invention and the enzyme digests thereof.

FIG. 9 shows neurite outgrowth promoting activities of skin-derived chondroitin sulfate/dermatan sulfate hybrid chains of the present invention and the enzyme digests thereof.

FIG. 10 shows effects of antibodies on neurite outgrowth promoting activities of a variety of neurotrophic factors (HGF, PTN, MK, and bFGF) and their receptors. Symbols “A” and “B” denote SL-CS/DS and E-CS/DS, respectively.

FIG. 11 shows effects of antibodies on neurite outgrowth promoting activities of a variety of neurotrophic factors (BDNF and GDNF) and their receptors. Symbols “A” and “B” denote SL-CS/DS and E-CS/DS, respectively.

### DESCRIPTION OF THE PREFERRED EMBODIMENTS

The present invention relates to novel oligosaccharides, chondroitin sulfate/dermatan sulfate hybrid chains, or chondroitin sulfate C. In the present invention, such chondroitin sulfate/dermatan sulfate hybrid chain and chondroitin sulfate C are referred to as polysaccharides in some cases. The animal species, from which the oligosaccharides, chondroitin sulfate/dermatan sulfate hybrid chains, or chondroitin sulfate C of the present invention are derived, are not limited. Examples of such animal species include mammals, birds, reptiles, amphibians, fish, chordates, molluscs, and arthropods.

Preferably, the oligosaccharides of the present invention can be obtained from a chondroitin sulfate/dermatan sulfate hybrid chains or chondroitin sulfate C derived from sharks. Preferably, the oligosaccharides of the present invention are oligosaccharides that can be obtained from chondroitin sulfate/dermatan sulfate hybrid chains derived from shark skin or chondroitin sulfate C derived from shark cartilage by the aforementioned methods. In addition, preferably, the chondroitin sulfate/dermatan sulfate hybrid chains of the present invention are chondroitin sulfate/dermatan sulfate hybrid chains that can be obtained from a shark liver and the chondroitin sulfate C that can be obtained from shark cartilage.

The oligosaccharides of the present invention are a sulfated oligosaccharides and preferably oligosaccharides having a hexasaccharide structure. An example of the oligosaccharides of the present invention are sulfated hexasaccharides that are obtained from a chondroitin sulfate/dermatan sulfate hybrid chain derived from shark skin, such a hexasaccharide represented as ΔHeXUAα1-3GalNAc(4S)β1-4IdoUA(2S)α1-3GalNAc(4S)β1-4IdoUA(2S)α1-3GalNAc(4S). The present invention encompasses chondroitin sulfate/dermatan sulfate hybrid chains having such oligosaccharide structures.

other examples of the oligosaccharides of the present invention are sulfated hexasaccharides that are obtained from chondroitin sulfate C derived from shark cartilage and have a disaccharide structure represented as A [GlcUAβ1-3GalNAc(4S)], B [GlcUA(2S)β1-3GalNAc(4S)], C [GlcUAβ1-3GalNAc(6S)], D [GlcUA(2S)β1-3GalNAc(4S)], or E [GlcUAβ1-3GalNAc(4S,6S)]. Specific examples thereof include tetrasulfated hexasaccharides, pentasulfated hexasaccharides, and hexasulfated hexasaccharides, which are represented as follows with the use of the aforementioned disaccharide structures A to E:

tetrasulfated hexasaccharides represented as ΔA-A-D, ΔC-D-C, ΔA-D-A, ΔA-D-C, or ΔC-A-D;

pentasulfated hexasaccharides represented as ΔA-D-D, ΔC-D-D, ΔA-B-D, ΔE-A-D, ΔD-D-C, ΔE-D-C, or ΔA-E-D; and

hexasulfated hexasaccharides represented as ΔD-D-D, ΔE-D-D, ΔA-D-T, or ΔD-A-T.

The present invention encompasses chondroitin sulfate C comprising such oligosaccharide structures.

The oligosaccharides of the present invention are chondroitinase-resistant oligosaccharides that are not degraded with chondroitinase AC-I.

In addition, the chondroitin sulfate/dermatan sulfate hybrid chains of the present invention are chondroitin sulfate/dermatan sulfate hybrid chains comprising at least one of disaccharide unit structures represented as Δ^{4,5}HexUAα1-3GalNAc(ΔO), GlcUAβ1-3GalNAc(O), Δ^{4,5}HexUAα1-3 GalNAc(4S)(ΔA), GlcUAβ1-3GalNAc(4S)(A), Δ^{4,5}HexUAα1-3GalNAc(6S)(ΔC), GlcUAβ1-3GalNAc(6S)(C), IdoUAα1-3GalNAc(6S)(iC), Δ^{4,5}HexUA(2S)α1-3GalNAc(4S)(ΔB), GlcUA(2S)β1-3GalNAc(4S)(B), IdoUA(2S)α1-3GalNAc(4S)(iB), Δ^{4,5}HexUA(2S)α1-3GalNAc(6S)(ΔD), GlcUA(2S)β1-3GalNAc(6S)(D), IdoUA(2S)α1-3GalNAc(6S)(iD), Δ^{4,5}HexUAα1-3GalNAc(4S,6S)(ΔE), GlcUAβ1-3GalNAc(4S,6S)(E), IdoUAα1-3GalNAc(4S,6S)(iE), Δ^{4,5} HexUA(2S)α1-3 GalNAc(4S,6S)(ΔT), and GlcUA(2S)β1-3 GalNAc(4S,6S)(T). The chondroitin sulfate/dermatan sulfate hybrid chain of the present invention may comprise at least one of the above disaccharide unit structures. There is no limitation in terms of the number of types of the above disaccharide units or the total number thereof. However, preferably, the chondroitin sulfate/dermatan sulfate hybrid chains of the present invention contain IdoUA(2S)α1-3GalNAc(4S) and/or GlcUA/IdoUAα1-3GalNAc(4S,6S) in large amounts. In addition, when a shark liver is subjected to hyaluronidase digestion, nitrous acid treatment, and desalting and the resulting fractions are introduced into anion-exchange resin followed by elution with a NaCl-containing eluent, a fraction eluted with NaCl (approximately 1.0 M) and a fraction eluted with NaCl (approximately 1.5 M) are obtained. The chondroitin sulfate/dermatan sulfate hybrid chains of the present invention may be a chondroitin sulfate/dermatan sulfate hybrid chain contained in either of such fractions. The average molecular weights of the former fraction and the latter fraction obtained via the measurement by gel filtration are approximately 75.7 kDa and approximately 3.8 to 38.9 kDa, respectively. In addition, the degree of sulfation of a disaccharide molecule contained in the chondroitin sulfate/dermatan sulfate hybrid chains of the present invention is less than 1.2, preferably approximately not less than 0.70 to less than 1.20, more preferably approximately not less than 0.80 to less than 1.20, particularly preferably approximately 0.87 to 1.17.

The aforementioned shark-derived oligosaccharide, chondroitin sulfate/dermatan sulfate hybrid chain, or chondroitin sulfate C can be separated and purified from shark body parts. There is no limitation in terms of such body parts. However, it is possible to adequately select the type of body part used in view of the content of the oligosaccharide, chondroitin sulfate/dermatan sulfate hybrid chain, or chondroitin sulfate C and the ease of separation/purification treatment. Examples of such body parts include shark skin, shark cartilage, and a shark liver. Glycosaminoglycan fractions obtained from samples by acetone extraction, protease treatment, and trichloroacetic acid extraction are further subjected to the steps of CPC (cetylpyridinium chloride) precipitation, hyaluronidase digestion, nitrous acid treatment, and desalting such that chondroitin sulfate/dermatan sulfate hybrid chain fractions or chondroitin sulfate C fractions can be obtained. The finally obtained chondroitin sulfate/dermatan sulfate hybrid chain fractions or chondroitin sulfate C fractions can be further fractionated by applying them to an anion-exchange resin and eluting the chondroitin sulfate/dermatan sulfate hybrid chains or chondroitin sulfate C fractions adsorbed on the resin with an NaCl-containing eluent. Examples of the chondroitin sulfate/dermatan sulfate hybrid chain of the present invention include chondroitin sulfate/dermatan sulfate hybrid chains that differ from one another depending on NaCl concentrations used for elution. Examples of chondroitin sulfate/dermatan sulfate hybrid chains include a chondroitin sulfate/dermatan sulfate hybrid chain contained in a fraction eluted with an eluent containing 1 M NaCl and that contained in a fraction eluted with an eluent containing 1.5 M NaCl. The obtained chondroitin sulfate/dermatan sulfate hybrid chain fractions or chondroitin sulfate C fractions are subjected to enzyme digestion with a variety of chondroitin sulfate catabolic enzymes such as chondroitinase AC-I. The resulting digests are applied to a HiTrap N-Hydroxysuccinimide-activated column (PTN column) which is a heparin-binding growth factor (namely, pleiotrophin (PTN)) coupling column, followed by elution. Thus, sulfated hexasaccharide fractions with high levels of PTN binding properties can be obtained.

When using animals other than sharks, separation/purification of chondroitin sulfate/dermatan sulfate hybrid chain fractions or chondroitin sulfate C fractions and separation/purification of oligosaccharide fractions derived from either thereof may be carried out by steps for separation/purification of methods known to persons skilled in the art. Examples of embodiments of a series of such steps are described in Examples below. However, the embodiments are not limited to the methods described below.

The oligosaccharide, the chondroitin sulfate/dermatan sulfate hybrid chain, and the chondroitin sulfate C of the present invention bonded with high affinity to a variety of proliferation factors (growth factors), cytokines (e.g., hepatocellular growth factors (HGF), RANTES (regulated upon activation, normal T cell expressed and secreted), fibroblast growth factor (FGF)-7, FGF-1, FGF-2, heparin-binding epidermal cell growth factor (HB-EGF), midkine (MK), pleiotrophin (PTN), and vascular endothelial growth factor (VEGF)).

Further, the oligosaccharide, the chondroitin sulfate/dermatan sulfate hybrid chain, and the chondroitin sulfate C of the present invention exhibit neurite outgrowth-promoting activity.

Specifically, the oligosaccharide, the chondroitin sulfate/dermatan sulfate hybrid chain, and the chondroitin sulfate C of the present invention bind to the aforementioned endogenous growth factors and cytokines so as to control activities of such growth factors and cytokines. That is, the oligosaccharide, the chondroitin sulfate/dermatan sulfate hybrid chain, and the chondroitin sulfate C of the present invention bind to the aforementioned endogenous growth factors and cytokines so as to control signal transduction in signal transduction systems of the above growth factors and cytokines, resulting in promotion of activities of such growth factors and cytokines.

The oligosaccharide, the chondroitin sulfate/dermatan sulfate hybrid chain, and the chondroitin sulfate C of the present invention are useful as therapeutic or preventive agents for neurodegenerative diseases and for the promotion of nervous system development by virtue of such properties. Further, they are also useful as therapeutic or preventive agents for treatment of inflammatory diseases or wounds.

Thus, the present invention also encompasses growth factor or cytokine (e.g., hepatocyte growth factors (HGF), RANTES (regulated upon activation, normal T cell expressed and secreted), fibroblast growth factor (FGF)-7, FGF-1, FGF-2, heparin-binding epidermal cell growth factor (HB-EGF), midkine (MK), pleiotrophin (PTN), or vascular endothelial growth factor (VEGF))-binding agents, neurite outgrowth-promoting agents, and anticoagulants, each comprising the aforementioned oligosaccharide, chondroitin sulfate/dermatan sulfate hybrid chain, or chondroitin sulfate C. Those skilled in the art can easily accomplish methods of production of these agents. For example, an oligosaccharide, chondroitin sulfate/dermatan sulfate hybrid chain, or chondroitin sulfate C may be completely or partially purified from animal body parts such as shark skin as described above, and the obtained fractions may be mixed, if necessary, with appropriate carriers and/or solvents.

The aforementioned oligosaccharide, chondroitin sulfate/dermatan sulfate hybrid chain, or chondroitin sulfate C is available as a pharmaceutical composition.

In particular, the above oligosaccharide, chondroitin sulfate/dermatan sulfate hybrid chain, or chondroitin sulfate C is available as a pharmaceutical composition for prevention or treatment of neurological diseases, inflammatory diseases, or wounds by virtue of its neurite outgrowth-promoting activity. Since neurological diseases are accompanied by the denaturation and loss of neurites in neurons, the oligosaccharide, chondroitin sulfate/dermatan sulfate hybrid chain, or chondroitin sulfate C is capable of preventing such events or promoting the outgrowth of new neurites, resulting in alleviation of the progression of symptoms of diseases or the alleviation of such symptoms. Examples of such neurological diseases include neurodegenerative diseases, including Alzheimer's disease and Parkinson's disease.

For these formulations or pharmaceutical compositions, it is preferred that the aforementioned oligosaccharide, chondroitin sulfate/dermatan sulfate hybrid chain, or chondroitin sulfate C should finally act at a concentration of 1 to 20 μg/ml, preferably 1 to 10 μg/ml, and more preferably 1 to 5 μg/ml.

For instance, when the oligosaccharide, chondroitin sulfate/dermatan sulfate hybrid chains, chondroitin sulfate C, or a fraction containing any thereof is applied as a neurite outgrowth-promoting agent, it is possible to administer a fraction obtained by completely or partially purifying the oligosaccharide, chondroitin sulfate/dermatan sulfate hybrid chain, or chondroitin sulfate C from shark body parts as described above. In such case, administration should be carried out in a manner such that such fraction is allowed to come into contact with neurons of target individuals in need of promotion of neurite outgrowth. An appropriate administration method can be selected by those skilled in the art. The above agent may be administered locally so that it may be delivered to neurons for which neurite elongation is desirable, or to the vicinity of such neurons. The agent should be delivered at a concentration of approximately 1 to 10 μg/ml, more preferably approximately 1 to 5 μ/ml, and further preferably approximately 2 μg/ml.

Further, when the oligosaccharide, chondroitin sulfate/dermatan sulfate hybrid chain, chondroitin sulfate C, or a fraction containing any thereof is applied as a growth factor or a cytokine-binding agent, the oligosaccharide, chondroitin sulfate/dermatan sulfate hybrid chain, chondroitin sulfate C, or a fraction containing any thereof in an amount appropriate for the relevant purpose (which differs depending on target growth factors or cytokines) may be mixed with growth factors so as to come into contact therewith.

Furthermore, when the oligosaccharide, chondroitin sulfate/dermatan sulfate hybrid chain, chondroitin sulfate C, or a fraction containing any thereof is applied as an anticoagulant, the oligosaccharide, chondroitin sulfate/dermatan sulfate hybrid chain, chondroitin sulfate C, or a fraction containing any thereof in an amount appropriate for the purpose may be mixed with growth factors so as to come into contact therewith.

The oligosaccharide, chondroitin sulfate/dermatan sulfate hybrid chain, or chondroitin sulfate C at a concentration of 0.1 μg or more and preferably 1 to 10 μg/ml is mixed with blood so as to come into contact therewith, resulting in exertion of its anticoagulant activity.

Moreover, it may be possible to use endogenous proliferation factors and/or endogenous cytokines contained in the oligosaccharide, chondroitin sulfate/dermatan sulfate hybrid chain, chondroitin sulfate C, or a fraction containing any thereof. Thus, the oligosaccharide, chondroitin sulfate/dermatan sulfate hybrid chains, chondroitin sulfate C, or a fraction containing any thereof may be administered alone.

Target individuals to which such agent is applied may be arbitrary mammals.

### EXAMPLES

The present invention is hereafter described in greater detail with reference to the following examples, although the technical scope of the present invention is not limited thereto.

### Example 1

### Separation of Polysaccharides and Oligosaccharides from Sharks Separation of Polysaccharides and Oligosaccharides from Shark Skin

Purification of chondroitin sulfate/dermatan sulfate hybrid chains from shark skin was carried out in accordance with an existing report (Nadini CD et al. (2005) J. Biol. Chem. 280, 4058-4069). Specifically, the skin of Prionace glauca was delipidated three times by acetone extraction and completely air-dried. The resulting product was suspended in water and then held in boiling water for 30 minutes for inactivation of protease.

The suspension was supplemented with borate-NaOH buffer and calcium chloride at final concentrations of 0.1 M and 10 mM, respectively, followed by digestion with protease (actinase; 2% by weight with respect to the weight of the sample) at 60° C. for 24 hours. After 24 hours and 48 hours, actinase (1% by weight with respect to the weight of the sample) was freshly added thereto for digestion. Then, 50% trichloroacetic acid was added thereto at a final concentration of 5%, followed by centrifugation for precipitation of proteins. The resultant was resuspended in 5% trichloroacetic acid, followed by additional centrifugation for collection of the supernatant.

Excessive trichloroacetic acid in the supernatant was removed by diethyl ether extraction. The resultant was allowed to stand overnight at 4° C. after addition of 4 times its volume of 80% ethanol containing 5% sodium acetate, resulting in the precipitation of glycosaminoglycan. The obtained precipitate was then collected by further centrifugation, followed by drying.

Purification of Glycosaminoglycan

The above glycosaminoglycan-containing precipitate was solubilized with 0.02 M Na₂SO₄, supplemented with 10% (w/v) cetylpyridinium chloride (CPC)/0.02 M Na₂SO₄, and allowed to stand overnight at room temperature. The obtained flocculent precipitate was redissolved with a 100:15 (v/v) 2 M NaCl/ethanol solution and precipitated again with the addition of 3 times its volume of 99.5% ethanol. The step described above was repeated three times, and precipitation in water and drying were finally performed. An additional CPC precipitation step was practiced in the same way by keeping a critical electrolyte concentration of NaCl at 0.5 M for removal of hyaluronic acid, resulting in the enrichment of glycosaminoglycan. Hyaluronidase digestion The CPC precipitate obtained above was dissolved in 0.02 M acetate buffer (pH 6) containing 0.15 M NaCl so as to be subjected to digestion with 50 TRU (turbidity reducing units) of Streptomyces hyaluronidase for 5 hours with shaking at 60° C. The resultant was further digested for 12 hours with the addition of 50 TRU of hyaluronidase. Then, the digestion of the hyaluronic acid was confirmed by cellulose acetate membrane electrophoresis. Thereafter, the digest was treated with TCA so as to remove proteins. Then, ethanol at a concentration of 64% was added thereto, resulting in precipitation of glycosaminoglycan.

Nitrous Acid Treatment

The precipitate obtained after hyaluronidase digestion was treated by allowing it to stand at room temperature for 40 minutes with the use of nitrous acid obtained by mixing equal volumes of 0.5 mmole of sulfuric acid and 0.5 mmole of barium nitrite. An aliquot of freshly prepared nitrous acid was added again thereto and the resultant was allowed to stand for 40 minutes. The treated sample was neutralized with 0.5 M Na₂CO₃ and desalted with a Sephadex G-50 column (1×56 cm) (eluent: 0.2 M ammonium bicarbonate; flow rate: 0.6 ml/min). The elution was monitored at 210 nm. The obtained eluted fractions were collected and subjected to repetitive procedures of lyophilization and reconstitution in water.

Purification of a Chondroitin Sulfate C Fraction with C18 and Anion-Exchange Columns

The desalted glycosaminoglycan was allowed to pass through a Sep-Pak C18 cartridge column and eluted with water and subsequently with 100% methanol. Further, the eluent was allowed to pass through an anion-exchange cartridge and eluted stepwise with 300 mM phosphate buffer containing 0.15, 0.5, 1.0, 1.5, or 2.0 M NaCl, followed by desalting in a PD-10 column with 50 mM pyridine acetate buffer (pH 5.0) as an eluent.

As a result of purification, a fraction eluted with the buffer containing NaCl contained chondroitin sulfate/dermatan sulfate hybrid chains.

The obtained chondroitin sulfate/dermatan sulfate hybrid chains were applied to a HiTrap N-Hydroxysuccinimide-activated column (PTN column) which is a pleiotrophin coupling column, such pleiotrophin serving as a heparin-binding growth factor.

The eluted 4 fractions were subjected to analysis of chondroitinase ABC-digested products, followed by disaccharide composition analysis. Table 1 shows the results.

TABLE 1

| | Subfractions | | | |
|---|---|---|---|---|
| | U-SS- | L-SS- | M-SS- | H-SS- |
| | CS/DS | CS-DS | CS/DS | CS/DS |
| Unsaturated disaccharides | mol % | | | |
| ΔO[ΔHexUA-GalNAc] | 27.9 | 7.8 | 6.4 | 4.7 |
| ΔC[ΔHexUA-GalNAc(6S)] | 36.5 | 34.7 | 16.6 | 11.2 |
| ΔA[ΔHexUA-GalNAc(4S)] | 30.7 | 44.1 | 56.3 | 60.3 |
| ΔD[ΔHexUA(2S)-GalNAc(6S)] | 1.1 | 3.6 | 3.4 | 2.1 |
| ΔB[ΔHexUA(2S)-GalNAc(4S)] | 0.2 | 1.3 | 8.8 | 13.3 |
| ΔE[ΔHexUA-GalNAc(6S,4S)] | 3.6 | 8.5 | 8.5 | 8.5 |
| Degree of sulfation | 0.77 | 1.06 | 1.14 | 1.19 |
| Subfraction percentage (%) | 23.3 | 51.7 | 21.9 | 3.1 |

Among the 4 fractions listed in table 1, the fraction (M-SS-CS/DS) to which pleiotrophin had been moderately bound was subjected to enzyme digestion with the use of chondroitinase AC-I. The obtained digest was applied to a PTN column. A chondroitinase AC-I-resistant fraction eluted with 0.15 M NaCl Tris-HCl buffer was subjected to saccharide composition analysis. Table 2 shows the results.

TABLE 2

| | Oligosaccharide | | |
|---|---|---|---|
| | Hexa | Octa | Deca |
| Unsaturated disaccharides | mol % | | |
| ΔO[ΔHexUA-GalNAc] | 5.3 | 6.4 | 5.9 |
| ΔC[ΔHexUA-GalNAc(6S)] | 20.1 | 15.1 | 13.9 |
| ΔA[ΔHexUA-GalNAc(4S)] | 38.0 | 47.7 | 54.1 |
| ΔD[ΔHexUA(2S)-GalNAc(6S)] | 6.3 | 6.7 | 5.9 |
| ΔB[ΔHexUA(2S)-GalNAc(4S)] | 17.7 | 13.4 | 11.3 |
| ΔE[ΔHexUA-GalNAc(6S,4S)] | 12.6 | 10.7 | 8.9 |

Further, anion-exchange HPLC and 500-MHz H-NMR spectroscopy were performed to analyze the structure of a chondroitinase AC-I-resistant hexasaccharide fraction obtained via purification following digestion of shark skin with chondroitinase AC-I.

As a result, a sulfated hexasaccharide having a novel structure represented as ΔHexUAα1-3 GalNAc(4S)β1-4IdoUA(2S)α1-3GalNAc(4S)β1-4IdoUA(2S)α1-3GalNAc(4S) was found.

Separation of Polysaccharides and Oligosaccharides from Shark Cartilage

In addition, separation, purification, and saccharide analysis were carried out as descried above with the use of oligosaccharides derived from shark cartilage-derived chondroitin sulfate C fractions.

As a result, disaccharide constituting the above hexasaccharide was found as A [GlcUAβ1-3GalNAc(4S)], B [GlcUA(2S)β1-3GalNAc(4S)], C [GlcUAβ1-3GalNAc(6S)], D [GlcUA(2S)β1-3GalNAc(4S)], or E [GlcUAβ1-3GalNAc(4S,6S)].

Tetrasulfated hexasaccharide, pentasulfated hexasaccharide, and hexasulfated hexasaccharide having the following structures were found:

tetrasulfated hexasaccharide having a structure represented as ΔA-A-D, ΔC-D-C, ΔA-D-A, ΔA-D-C, or ΔC-A-D;

pentasulfated hexasaccharide having a structure represented as ΔA-D-D, ΔC-D-D, ΔA-B-D, ΔE-A-D, ΔD-D-C, ΔE-D-C, or ΔA-E-D; and

hexasulfated hexasaccharide having a structure represented as ΔD-D-D, ΔE-D-D, ΔA-D-T, or ΔD-A-T.

Separation of Polysaccharides from Shark Liver

A chondroitin sulfate/dermatan sulfate hybrid fraction was separated from shark liver and purified by a method similar to the aforementioned method. (When separation and purification was carried out using anion-exchange resin, a fraction eluted with 1.0 M NaCl buffer and a fraction eluted with 1.5 M NaCl buffer were designated as SL-CS/DS (1.0 M) and SL-CS/DS (1.5 M), respectively.)

Table 3 shows results of analysis of disaccharides constituting a chondroitin sulfate/dermatan sulfate hybrid subjected to enzyme digestion with the use of chondroitinases ABC, B, and AC-I.

TABLE 3

| | SL-CS/DS (1.0 M) | | | SL-CS/DS (1.5 M) | | |
|---|---|---|---|---|---|---|
| | | | AC-I | | | AC-I |
| Unsaturated | ABC | B | and II | ABC | B | and II |
| disaccharide | pmol (mol %) | | | | | |
| ΔDi-0S | 15.5 | — | 11.2 | 6.9 | — | 2.2 |
| | (6.3) | | (11.6) | (2.6) | | (6.4) |
| ΔDi-6S | 78.1 | 5.6 | 25.0 | 11.7 | — | 4.9 |
| | (31.8) | (9.0) | (25.8) | (4.3) | | (13.9) |
| ΔDi-4S | 81.4 | 39.8 | 38.6 | 132.7 | 49.7 | 13.8 |
| | (33.1) | (64.0) | (39.8) | (49.4) | (42.6) | (39.3) |
| ΔDi-diS_{D} | 10.5 | 1.0 | 3.0 | 2.8 | 0.5 | 0.3 |
| | (4.3) | (1.6) | (3.1) | (1.0) | (0.4) | (1.0) |
| ΔDi-diS_{B} | 16.7 | 8.8 | 7.0 | 48.2 | 45.9 | 3.3 |
| | (6.8) | (14.1) | (7.2) | (18.0) | (39.4) | (9.3) |
| ΔDi-diS_{E} | 43.4 | 7.0 | 12.1 | 61.2 | 15.2 | 10.6 |
| | (17.7) | (11.3) | (12.5) | (22.8) | (13.0) | (30.2) |
| ΔDi-triS | — | — | — | 4.9 | 5.4 | — |
| | | | | (1.8) | (4.6) | |
| Total | 245.6 | 62.2 | 96.9 | 268.3 | 118.9 | 35.5 |
| S/unit^{b} | 1.18 | — | — | 1.43 | — | — |

ΔDi-0S, Δ^{4,5}HexUAα1-3GalNAc; ΔDi-4S, Δ^{4,5}HexUAα1-3GalNAc(4S); ΔDi-6S, Δ^{4,5}HexUAα1-3GalNAc(6S); ΔDi-diS_{B}, Δ^{4,5}HexUA(2S) α1-3GalNAc(4S); ΔDi-diS_{D}, Δ^{4,5}HexUA(2S) α1-3GalNAc(6S); ΔDi-diS_{E}, Δ^{4,5}HexUAα1-3GalNAc(4S,6S)

As shown in table 3, the chondroitin sulfate/dermatan sulfate hybrid was found to have a disaccharide composition rich in IdoUA(2S)α1-3GaINAc(4S) and GlcUA/IdoUAα1-3GalNAc(4S,6S).

### Example 2

### Analysis of Interaction between a Variety of Proliferation Factors and Oligosaccharides or Polysaccharides

Interaction between a Variety of Proliferation Factors and Oligosaccharides

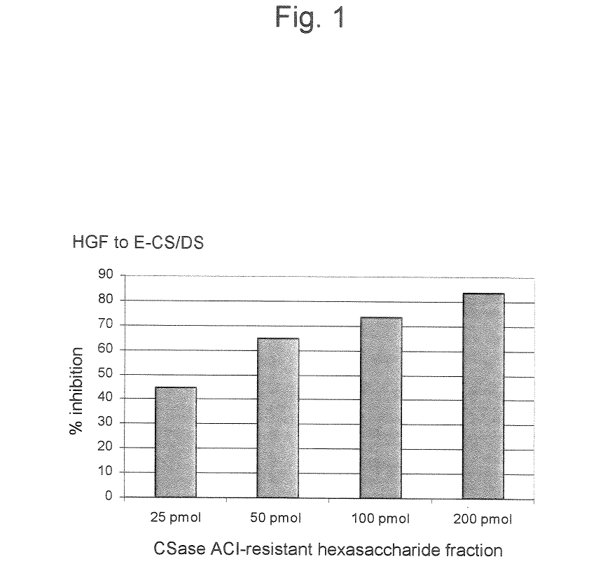

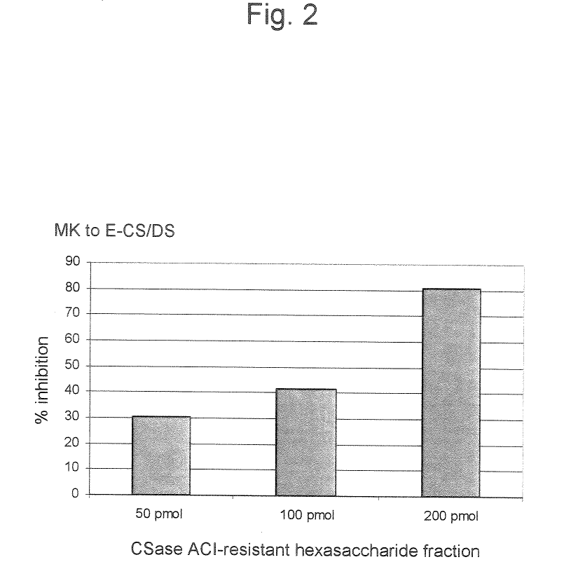

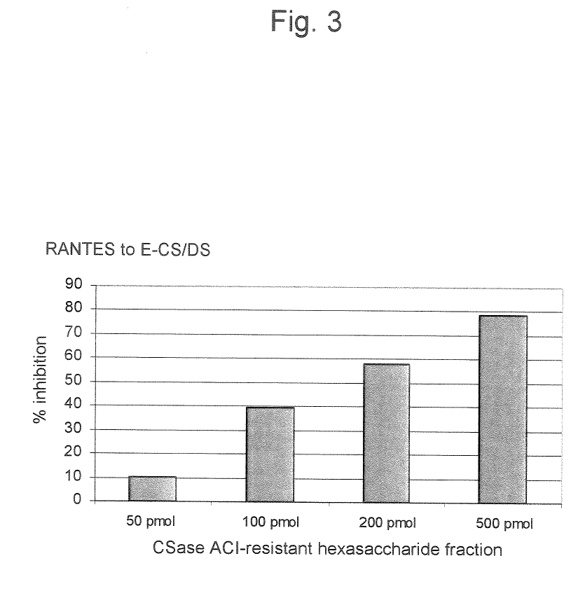

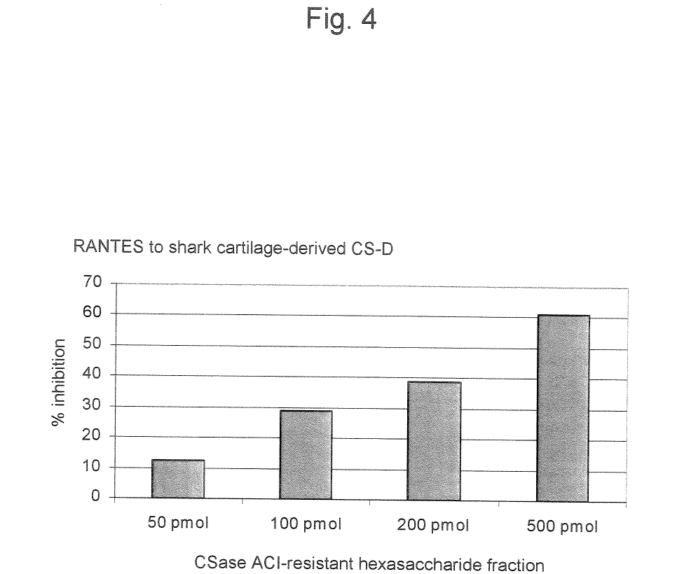

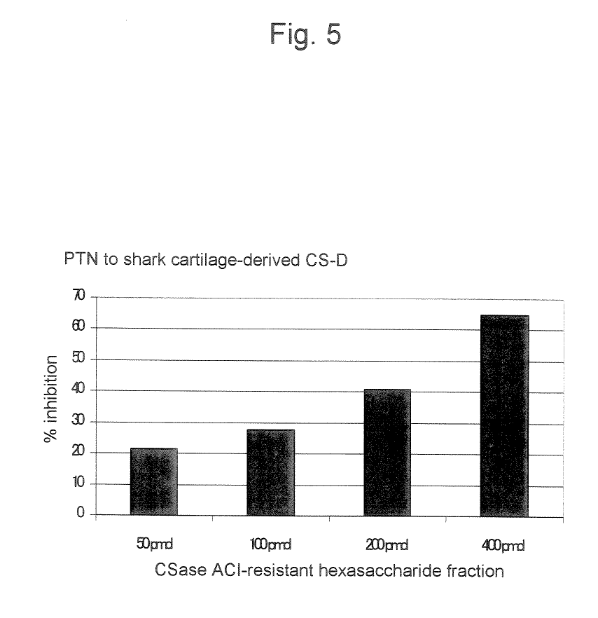

Interaction between chondroitinase AC-I-resistant sulfated hexasaccharide derived from the aforementioned shark cartilage chondroitin sulfate C and growth factors or cytokines was analyzed. A BIAcore J system was used for analysis. Sulfated hexasaccharide was evaluated based on activity of inhibiting binding of growth factors or cytokines to CS-D or embryonic pig brain-derived CS/DS (E-CS/DS). Chondroitinase AC-I-resistant sulfated hexasaccharide was separately mixed with HGF (hepatocyte growth factor), MK, PTN, and RANTES (regulated upon activation, normal T cell expressed and secreted), followed by incubation at 37° C. for 30 minutes. Each mixed solution was allowed to react on a sensor chip to which CS-D or E-CS/DS had been bound. The rate of reaction inhibition was examined. FIGS. 1 to 5 show the results. As shown in FIGS. 1 to 5, it was revealed that sulfated hexasaccharide had strong binding inhibiting activity against binding of any growth factor or cytokine to CS-D or E-CS/DS.

Interaction between Various Proliferation Factors and Polysaccharides and the Enzyme Digests thereof

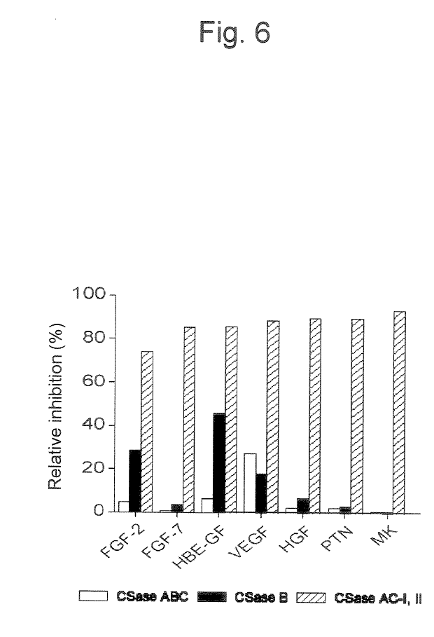

Interaction between chondroitin sulfate/dermatan sulfate hybrid fractions separated and purified from shark liver and a variety of growth factors or cytokines (FGF-2, -7, HB-EGF, VEGF, HGF, PTN, and MK) was examined by a method similar to the aforementioned method. FIG. 6 shows the results.

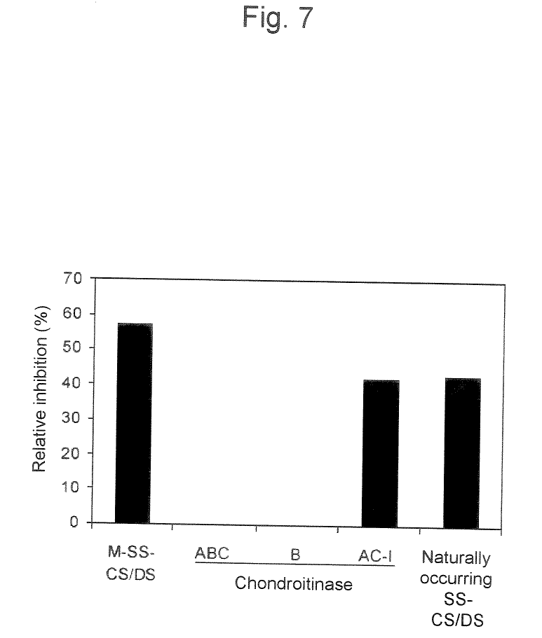

As shown in FIG. 6, chondroitin sulfate/dermatan sulfate hybrid chondroitinase degradation products were found to have high affinity with various growth factors or cytokines. Interaction between PTN and polysaccharides and the enzyme digests thereof Interaction between PTN and chondroitin sulfate/dermatan sulfate hybrid chains derived from shark skin and the chondroitinase AC-I-digested products thereof was analyzed. A BIAcore J system was used for analysis as described above. Evaluation was carried out using the shark skin-derived chondroitin sulfate/dermatan sulfate hybrid chains and the chondroitinase AC-I-digested products thereof based on activity of inhibiting binding of PTN to E-CS/DS. FIG. 7 shows the results. As shown in FIG. 7, it was revealed that resistant oligosaccharides contained in shark skin-derived chondroitin sulfate/dermatan sulfate hybrid chains and the chondroitinase AC-I-digested products thereof have strong binding inhibiting activity against binding of PTN to E-CS/DS.

### Example 3

### Examination of Neurite Outgrowth Promoting Activity of Chondroitin Sulfate/Dermatan Sulfate Hybrid Chain

In accordance with the method of a past report (Hikino et al. 2003 J. Biol Chem 278, 43744-43754), neurite outgrowth promoting activity was examined with the use of cultured neurons.

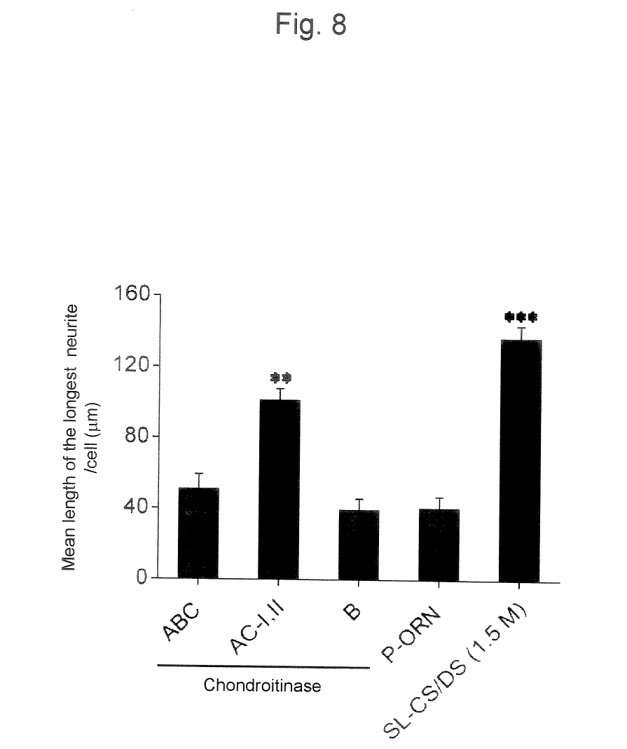

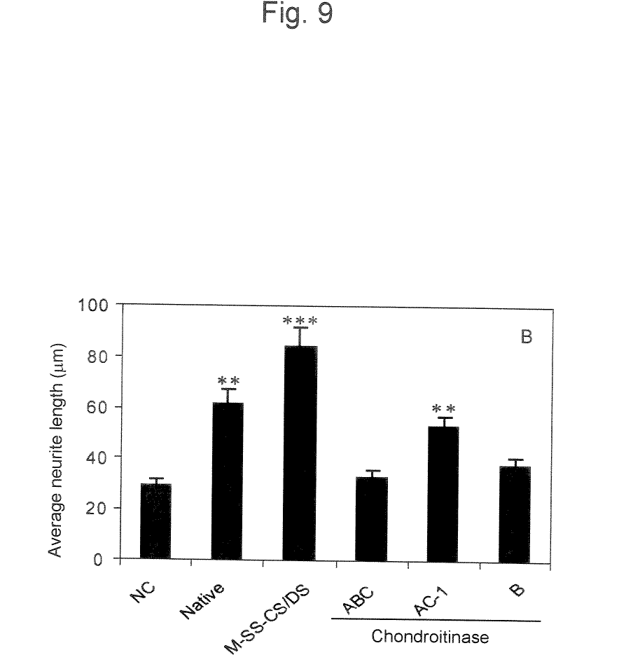

That is, a preparation was digested with chondroitinase ABC, AC-I, or B. Then, the resulting digest was added to a culture solution. After culture at 37° C. for 24 hours, cells were immobilized, followed by immunostaining with specific antibodies. Then, lengths of neurites were measured under a microscope. FIGS. 8 and 9 show the results.

As shown in FIG. 8, liver-derived chondroitin sulfate/dermatan sulfate hybrid chains exhibited high neurite outgrowth promoting activity. In addition, resistant oligosaccharides in chondroitinase digests maintained their activities. In particular, the AC-I,II-digested product exhibited activity comparable to those of polysaccharides.

Further, as shown in FIG. 9, skin-derived chondroitin sulfate/dermatan sulfate hybrid chains exhibited high neurite outgrowth-promoting activity. In addition, resistant oligosaccharides in the chondroitinase AC-I-digested products maintained their activities.

### Example 4

### Examination of a Neurite Outgrowth Promoting Activity Control Mechanism of SL-CS/DS (1.5 M)

Coverslips that had been coated with poly-DL-ornithine (P-ORN) (Sigma) were placed on a microplate. Then, SL-CS/DS (1.5 M) was immobilized thereon in an amount of 2 μg per well.

Hippocampal neurons that had been established from an E16 mouse were dispersed in Eagle's MEM. The neurons were seeded on the coverslips at a density of 10,000 cells/cm², followed by culture at 37° C. in the presence of 5% CO₂.

Upon measurement of neutralizing activity, anti-PTN antibodies, anti-MK antibodies, anti-bFGF antibodies, anti-HGF antibodies, anti-BDNF antibodies, and anti-GDNF antibodies were added to culture solutions at 10 μg/ml, 10 μg/ml, 10 μg/ml, 3 μg/ml, 5 μg/ml, and 5 μg/ml, respectively, 2 hours after the seeding of cells.

After overnight culture, cells on each coverslip were immobilized at room temperature for 30 minutes with the use of 4% (w/v) paraformaldehyde. The thus immobilized neurons were subjected to immunostaining with the use of monoclonal antibodies against neurofilament and microtubule-related proteins.

The immunostained cells on each coverslip were subjected to digital photography under an optical microscope. Cells that had been randomly selected were subjected to measurement of the maximum lengths of the neurites and the determination of the number thereof with the use of image analysis software (Mac SCOPE; Mitani Corporation, Tokyo, Japan).

It has been known that HGF, MK, PTN, bFGF, BDNF, and GDNF act on cells via an autocrine or paracrine mechanism. CS/DS has a high ability of binding to these nutritional factors. Thus, it was predicted that CS/DS would interact with nutritional factors secreted by neurons and would be involved in nerve growth (J. Biol. Chem. 279, 50799-50809, 2004).

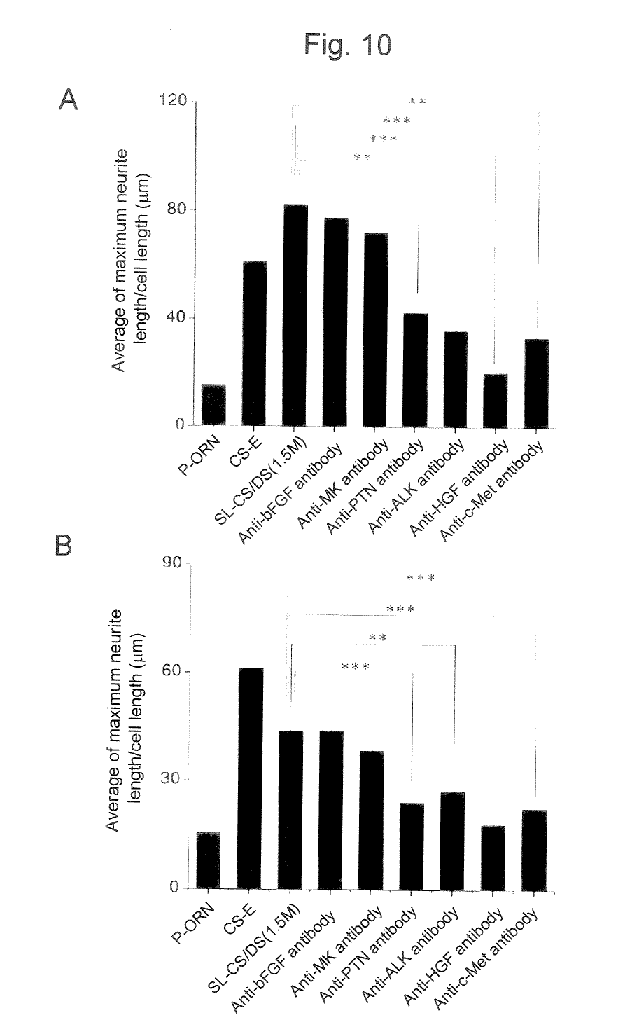

Neutralization tests were conducted with the use of antibodies against a variety of nutritional factors. As a result, in the cases of cells to which anti-HGF antibodies, anti-PTN antibodies, anti-BDNF antibodies, and anti-GDNF antibodies had been added, neurite outgrowth promotion (NOP) activity of SL-CS/DS (1.5 M) was obviously suppressed compared with the case of a group to which antibodies had not been added. On the contrary, in the cases of cells to which anti-MK polyclonal antibodies and anti-bFGF monoclonal antibodies had been added, such NOP-suppressing effect was not observed (FIGS. 9A and 10A).

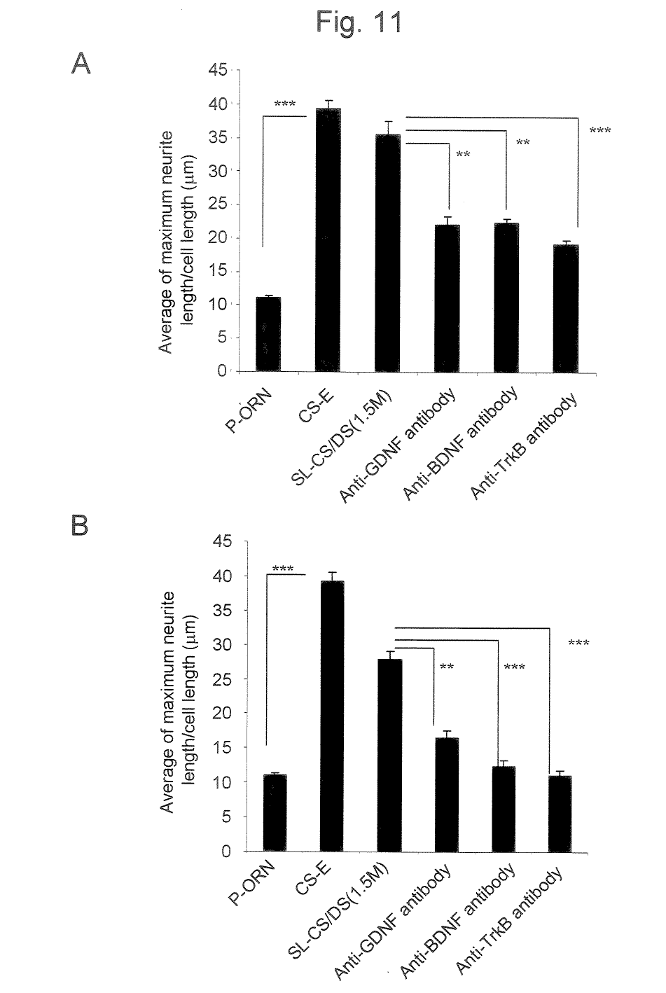

Further, also in the cases in which antibodies against an HGF receptor “c-Met,” a PTN receptor “ALK,” and a BDNF receptor “TrkB” had been added, NOP activity of SL-CS/DS (1.5 M) was obviously suppressed compared with the case of a group to which no antibodies had been added (FIGS. 10A and 11A).

In addition, embryonic pig-derived CS/DS chains (E-CS/DS) were subjected to a test similar to that used in the case of SL-CS/DS (1.5 M). Accordingly, results similar to those obtained in the case of SL-CS/DS (1.5 M) were obtained as expected (FIGS. 10B and 11B).

Based on the above results, it has been suggested that neurite outgrowth promoting activity of SL-CS/DS (1.5M) is controlled selectively via signal transduction systems of HGF, PTN, BDNF, and GDNF.

What is claimed is:

1. Oligosaccharides derived from chondroitin sulfate/dermatan sulfate hybrid chains, comprising sulfated hexasaccharide structures and being resistant to chondroitinase AC-I, wherein the oligosaccharides are derived from a chondroitin sulfate/dermatan sulfate hybrid.

2. The oligosaccharides according to claim 1, wherein the oligosaccharides are derived from chondroitin sulfate/dermatan sulfate hybrid chains extracted from shark skin.

3. The oligosaccharides derived from chondroitin sulfate/dermatan sulfate hybrid chains according to claim 1 or 2, wherein the oligosaccharides have neurite outgrowth-promoting activity and bind to pleiotrophin.

4. Oligosaccharides derived from chondroitin sulfate/dermatan sulfate hybrid chains, wherein the oligosaccharides are represented as ΔHexUAα1-3GalNAc(4S)β1-4IdoUA(2S)α1-3GalNAc(4S)β1-4IdoUA(2S)α1-3GalNAc(4S).

5. A chondroitin sulfate/dermatan sulfate hybrid chain comprising a structure represented as ΔHexUAα1-3GalNAc(4S)β1-4IdoUA(2S)α1-3GalNAc(4S)β1-4IdoUA(2S)α1-3 GalNAc(4S).

6. Oligosaccharides derived from chondroitin sulfate C, comprising a sulfated hexasaccharide structure and being resistant to chondroitinase AC-I.

7. The oligosaccharides according to claim 6, wherein the oligosaccharides are derived from chondroitin sulfate C extracted from shark cartilage.

8. The oligosaccharidse derived from chondroitin sulfate C according to claim 6 or 7, wherein the oligosaccharides bind to hepatocellular growth factors, midkine, pleiotrophin, and RANTES.

9. An oligosaccharide derived from chondroitin sulfate C, comprising a structure selected from the group consisting of ΔHexUAα1-3GalNAc(4S)β1-4GlcUAβ1-3GalNAc(4S)β1-4GlcUA(2S)β1-3GalNAc(6S)(ΔA-A-D), ΔHexUAα1-3GalNAc(6S)β1-4GlcUA(2S)β1-3GalNAc(6S)β1-4GlcUAβ1-3GalNAc(6S) (ΔC-D-C), ΔHexUAα1-3GalNAc(4S)β1-4GlcUA(2S)β1-3GalNAc(6S)β1-4GlcUAβ1-3GalNAc(4S) (ΔA-D-A), ΔHexUAα1-3GalNAc(4S)β1-4GlcUA(2S)α1-3GalNAc(6S)β1-4GlcUA(2S)β1-3GalNAc(6S) (ΔA-D-D), ΔHexUAα1-3GalNAc(6S)β1-4GlcUA(2S)β1-3GalNAc(6S)β1-4GlcUA(2S)β1-3GalNAc(6S) (ΔC-D-D), ΔHexUAα1-3GalNAc(4S)β1-4GlcUA(2S)β1-3GalNAc(6S)β1-4GlcUAβ1-3GalNAc(6S) (ΔA-D-C), ΔHexUAα1-3GalNAc(4S,6S)β1-4GlcUAβ1-3GalNAc(4S)β1-4GlcUA(2S)β1-3 GalNAc(6S) (ΔE-A-D), ΔHexUA(2S)α(1-3GalNAc(6S)β1-4GlcUA(2S)β1-3GalNAc(6S)β1-4GlcUAβ1-3GalNAc(6S) (ΔD-D-C), ΔHexUAα1-3GalNAc(4S,6S)β1-4GlcUA(2S)β1-3GalNAc(6S)β1-4GlcUAβ1-3GalNAc(6S) (ΔE-D-C), ΔHexUAα1-3GalNAc(6S)β1-4GlcUA1-3GalNAc(4S)β1-4GlcUA(2S)β1-3GalNAc(6S) (ΔC-A-D), ΔHexUAα1-3GalNAc(4S)β1-4GlcUAβ1-3GalNAc(4S,6S)β1-4GlcUA(2S)β1-3GalNAc(6S) (ΔA-E-D), ΔHexUAα1-3GalNAc(4S)β1-4GlcUA(2 S)β1-3 GalNAc(4S)β1-4GlcUA(2S)β1-3GalNAc(6S) (ΔA-B-D), ΔHexUA(2S)α1-3GalNAc(6S)β1-4GlcUA(2S)β1-3GalNAc(6S)β1-4GlcUA(2S)β1-3GalNAc(6 S) (ΔD-D-D), ΔHexUAα1-3GalNAc(4S,6S)β1-4GlcUA(2S)β1-3GalNAc(6S)β1-4GlcUA(2S)β1-3GalNAc(6S) (ΔE-D-D), and ΔHexUAα1-3GalNAc(4S)β1-4GlcUA(2S)β1-3GalNAc(6S)β1-4GlcUA(2S)β1-3GalNAc(4S,6S) (ΔA-D-T) or ΔHexUA(2S)α1-3GalNAc(6S)β1-4GlcUAβ1-3GalNAc(4S)β1-4GlcUA(2S)β1-3GalNAc(4S,6S) (ΔD-A-T).

10. Chondroitin sulfate C comprising a structure of the oligosaccharide derived from chondroitin sulfate C according to claim 9.

11. Chondroitin sulfate/dermatan sulfate hybrid chains comprising at least one unit selected from the group consisting of Δ^{4,5}HexUAα1-3GalNAc(ΔO), GlcUAβ1-3GalNAc(O), Δ^{4,5}HexUAα1-3GalNAc(4S)(ΔA), GlcUAβ1-3GalNAc(4S)(A), Δ^{4,5}HexUAα1-3GalNAc(6S)(ΔC), GlcUAβ1-3GalNAc(6S)(C), IdoUAα1-3GalNAc(6S)(iC), Δ^{4,5}HexUA(2S)α1-3GalNAc(4S)(ΔB), GlcUA(2S)β1-3GalNAc(4S)(B), IdoUA(2S)α1-3GalNAc(4S)(iB), Δ^{4,5}HexUA(2S)α1-3GalNAc(6S)(ΔD), GlcUA(2S)β1-3GalNAc(6S)(D), IdoUA(2S)α1-3GalNAc(6S)(iD), Δ^{4,5}HexUAα1-3GalNAc(4S,6S)(ΔE), GlcUAβ1-3GalNAc(4S,6S)(E), IdoUAα1-3GalNAc(4S,6S)(iE), Δ^{4,5}HexUA(2S)α1-3GalNAc(4S,6S)(ΔT), and GlcUA(2S)β1-3GalNAc(4S,6S)(T).

12. The chondroitin sulfate/dermatan sulfate hybrid chains according to claim 11, wherein the chains are derived from shark liver.

13. The chondroitin sulfate/dermatan sulfate hybrid chains according to claim 11 or 12, wherein the chains have neurite outgrowth-promoting activity and bind to fibroblast growth factor-2, fibroblast growth factor-7, heparin-binding epidermal cell growth factor, vascular endothelial growth factor, hepatocellular proliferation factors, midkine, and pleiotrophin.

14. An agent for binding a growth factor or a cytokine comprising, as an active ingredient, at least one oligosaccharide, chondroitin sulfate/dermatan sulfate hybrid chain, or chondroitin sulfate C selected from the group consisting of the oligosaccharide according to any one of claims 1 to 4, the chondroitin sulfate/dermatan sulfate hybrid chain according to claim 5, the oligosaccharide according to any one of claims 6 to 9, the chondroitin sulfate C according to claim 10, and the chondroitin sulfate/dermatan sulfate hybrid chain according to any one of claims 11 to 13.

15. An agent for promoting a growth factor or cytokine activity comprising, as an active ingredient, at least one oligosaccharide, chondroitin sulfate/dermatan sulfate hybrid chain, or chondroitin sulfate C selected from the group consisting of the oligosaccharide according to any one of claims 1 to 4, the chondroitin sulfate/dermatan sulfate hybrid chain according to claim 5, the oligosaccharide according to any one of claims 6 to 9, the chondroitin sulfate C according to claim 10, and the chondroitin sulfate/dermatan sulfate hybrid chain according to any one of claims 11 to 13.

16. An agent for promoting neurite outgrowth comprising, as an active ingredient, at least one oligosaccharide, chondroitin sulfate/dermatan sulfate hybrid chain, or chondroitin sulfate C selected from the group consisting of the oligosaccharide according to any one of claims 1 to 4, the chondroitin sulfate/dermatan sulfate hybrid chain according to claim 5, the oligosaccharide according to any one of claims 6 to 9, the chondroitin sulfate C according to claim 10, and the chondroitin sulfate/dermatan sulfate hybrid chain according to any one of claims 11 to 13.

17. A pharmaceutical composition comprising, as an active ingredient, at least one oligosaccharide, chondroitin sulfate/dermatan sulfate hybrid chain, or chondroitin sulfate C selected from the group consisting of oligosaccharide according to any one of claims 1 to 4, chondroitin sulfate/dermatan sulfate hybrid chain according to claim 5, the oligosaccharide according to any one of claims 6 to 9, the chondroitin sulfate C according to claim 10, and the chondroitin sulfate/dermatan sulfate hybrid chain according to any one of claims 11 to 13.

18. A pharmaceutical composition for prevention or treatment of neurological diseases, inflammatory diseases, or wounds comprising, as an active ingredient, at least one oligosaccharide, chondroitin sulfate/dermatan sulfate hybrid chain, or chondroitin sulfate C selected from the group consisting of the oligosaccharide according to any one of claims 1 to 4, the chondroitin sulfate/dermatan sulfate hybrid chain according to claim 5, the oligosaccharide according to any one of claims 6 to 9, the chondroitin sulfate C according to claim 10, and the chondroitin sulfate/dermatan sulfate hybrid chain according to any one of claims 11 to 13.

19. Use of at least one oligosaccharide, chondroitin sulfate/dermatan sulfate hybrid chain, or chondroitin sulfate C selected from the group consisting of the oligosaccharide according to any one of claims 1 to 4, the chondroitin sulfate/dermatan sulfate hybrid chain according to claim 5, the oligosaccharide according to any one of claims 6 to 9, the chondroitin sulfate C according to claim 10, and the chondroitin sulfate/dermatan sulfate hybrid chain according to any one of claims 11 to 13 for production of a growth factor-binding agent, a neurite outgrowth-promoting agent, a composition for neurological disease treatment, a composition for inflammatory disease treatment, or a composition for wound treatment.

20. Use of at least one oligosaccharide, chondroitin sulfate/dermatan sulfate hybrid chain, or chondroitin sulfate C selected from the group consisting of the oligosaccharide according to any one of claims 1 to 4, the chondroitin sulfate/dermatan sulfate hybrid chain according to claim 5, the oligosaccharide according to any one of claims 6 to 9, the chondroitin sulfate C according to claim 10, and the chondroitin sulfate/dermatan sulfate hybrid chain according to any one of claims 11 to 13 for production of a growth factor activity-promoting agent.
